(19) United States
(12) Patent Application Publication
(10) Pub. No.: US 2017/0226072 A1
(43) Pub. Date: Aug. 10, 2017

(54) 1,4-Disubstituted Piperidines, 1,4-Disubstituted Piperazines, 1,4-Disubstituted Diazepines, and 1,3-Disubstituted Pyrrolidine Compounds

(71) Applicant: University of Kentucky Research Foundation, Lexington, KY (US)

(72) Inventors: Peter A. CROOKS, Little Rock, AR (US); Linda P. DWOSKIN, Lexington, KY (US); John CULVER, Richmond, KY (US); Justin R. NICKELL, Lexington, KY (US); Guangrong ZHENG, Little Rock, AR (US)

(21) Appl. No.: 15/494,789

(22) Filed: Apr. 24, 2017

Related U.S. Application Data
(62) Division of application No. 14/776,306, filed on Sep. 14, 2015, now abandoned.
(60) Provisional application No. 61/793,281, filed on Mar. 15, 2013.

(51) Int. Cl. C07D 295/096; C07D 295/073; C07D 211/22; C07D 211/18; C07D 211/14; C07D 295/03

(52) U.S. Cl. CPC C07D 295/096; C07D 211/14; C07D 295/03; C07D 211/22; C07D 211/18; C07D 295/073

(57) ABSTRACT

The present invention is directed to 1,4-disubstituted piperidines, 1,4-disubstituted piperazines, 1,4-disubstituted diazepanes, and 1,3-disubstituted pyrrolidine compounds and their use.

### CROSS-REFERENCE TO RELATED APPLICATIONS

This application is a divisional of U.S. application Ser. No. 14/776,306, filed Sep. 14, 2015, which is a 371 of International Application No. PCT/US2014/028315, filed Mar. 14, 2014, which claims the benefit to U.S. provisional Application No. 61/793,281, the contents of which are incorporated by reference herein.

### FIELD OF THE INVENTION

The present invention relates to 1,4-disubstituted piperidine, 1,4-disubstituted piperazine, 1,4-disubstituted diazepane, and 1,3-disubstituted pyrrolidine compounds, and their method of use in the treatment of diseases and pathologies of the central nervous system (CNS), the treatment of drug dependence/abuse and withdrawal therefrom, and the treatment of eating disorders such as obesity.

### BACKGROUND OF THE INVENTION

Alpha-Lobeline (lobeline), a lipophilic nonpyridino, alkaloidal constituent of Indian tobacco; is a major alkaloid in a family of structurally-related compounds found in Lobelia inflata. Lobeline (i.e., 2-[6-(β-hydroxyphenethyl)-1-methyl-2-piperidyl]-acetophenone) has been reported to have many nicotine-like effects, including tachycardia and hypertension (Olin et al., 1995), hyperalgesia (Hamann et al., 1994) and improvement of learning and memory (Decker et al., 1993). Lobeline has high affinity for nicotinic receptors (Lippiello et al., 1986; Broussolle et al., 1989). Differential effects of lobeline and nicotine suggest that these drugs may not be active through a common CNS mechanism, even though lobeline has been considered a mixed nicotinic agonist/antagonist.

Lobeline evokes dopamine (DA) release from rat striatal slices. However, lobeline-evoked DA release is neither dependent upon extracellular calcium nor is it sensitive to mecamylamine, a noncompetitive nicotinic receptor antagonist. Thus, lobeline-evoked DA release occurs via a different mechanism than does nicotine to evoke DA release (Teng et al., 1997, 1998; Clarke et al., 1996). In this respect, lobeline also inhibits DA uptake into rat striatal synaptic vesicles via an interaction with the dihydrotetrabenazine (DTBZ) site on vesicular monoamine transporter-2 (VMAT2), increasing the cytosolic DA available for reverse transport by the plasma membrane dopamine transporter (DAT) (Teng et al., 1997, 1998). Thus, lobeline interacts with nicotinic receptors and blocks nicotine-evoked DA release, but also interacts with DA transporter proteins (DAT and VMAT2) to modify the concentration of DA in the cytosolic and vesicular storage pools, thereby altering subsequent dopaminergic neurotransmission.

The action of many neuropharmacologically therapeutic agents involve the modulation of DA, norepinephrine (NE) and serotonin (5-HT) release, uptake and storage within their respective terminals in the central nervous system (CNS). Most neurotransmitters are stored in synaptic vesicles, which are prominent features of nerve terminals. Sequestration into vesicles appears to be responsible for maintaining a ready supply of neurotransmitter available for neuronal exocytotic release into the synaptic cleft. Vesicles also serve the role of protecting the neurotransmitter from metabolic breakdown. One transport site on the vesicle membrane is the vesicular monoamine transporter-2 (VMAT2), whose role is to transport transmitter from the cytosol into the synaptic vesicle. DTBZ, a ligand structurally related to methoxytetrabenazine (MTBZ), has been used as a radiolabel to probe the interaction of drugs with VMAT2. Both DTBZ and MTBZ act at the same site on VMAT2. Once the neurotransmitter is released from the terminal into the synaptic space, it interacts with postsynaptic receptors and subsequently is taken back up into the terminal via the plasma membrane transporter (e.g., DAT and/or the serotonin transporter [SERT]). Thus, transporter proteins modify the concentration of neurotransmitter in the cytosolic and vesicular storage pools, thereby having the ability to alter subsequent neurotransmission.

### BRIEF SUMMARY OF THE INVENTION

Disclosed herein is a compound of formula (I), without regard to chirality:

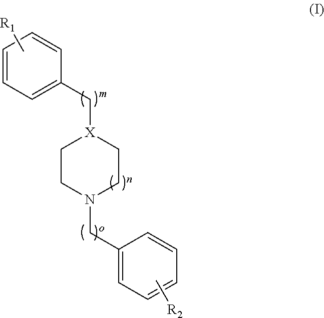

wherein

m is an integer in the range from 1 to 3;

n is zero or an integer in the range from 1 to 2;

o is an integer in the range from 1 to 3;

X represents either a nitrogen atom or a carbon atom with a single hydrogen attached; and

R₁ and R₂ are independently selected from the group consisting of hydrogen; methyl; deuteromethyl (CD₃); tritiomethyl (CT₃); ethyl; propyl; isopropyl; C₄-C₇ straight chain or branched alkyl; C₃-C₆ cycloalkyl; C₄-C₇ alkenyl (including cis and trans geometrical forms); benzyl; phenylethyl; amino; N-methylamino; N,N-dimethylamino; carboxylate; methylcarboxylate; ethylcarboxylate; propylcarboxylate; isopropylcarboxylate; carboxaldehyde; acetoxy; propionyloxy; isopropionyloxy; cyano; aminomethyl; N-methylaminomethyl; N,N-dimethylaminomethyl; carboxamide; N-methylcarboxamide; N,N-dimethylcarboxamide; acetyl; propionyl; formyl; benzoyl sulfate; phenyl; methylsulfate; hydroxyl; methoxy; ethoxy; propoxy; isopropoxy; thiol; methylthio; ethylthio; propiothiol; fluoro; chloro; bromo; iodo; trifluoromethyl; vinyl; allyl; propargyl; nitro; carbamoyl; ureido; azido; isocyanate; thioisocyanate; hydroxylamino; nitroso; a saturated or unsaturated hydrocarbon ring; a nitrogen containing heterocyclic moiety; an oxygen containing heterocyclic moiety; a sulfur containing heterocyclic moiety; a selenium containing heterocyclic moiety; a mixed heterocyclic moiety containing at least two atoms selected from the group consisting of nitrogen, oxygen and sulfur; and ortho, meta or para-substituted benzene,

or a pharmaceutically acceptable salt thereof

Also disclosed herein is a pharmaceutical composition comprising the compound of formula (I).

Further disclosed herein are the following methods: a method of treating an eating disorder in an individual in need thereof, comprising administering to the individual the compound of formula (I) according to claim 1 or a pharmaceutically acceptable salt thereof a method of treating a disease or pathology of the central nervous system in an individual in need thereof, comprising administering to the individual the compound of formula (I) according to claim 1 or a pharmaceutically acceptable salt thereof and a method of treating an individual for drug dependence/abuse or withdrawal from drug dependence/abuse, comprising administering to the individual the compound of formula (I) according to claim 1 or a pharmaceutically acceptable salt thereof.

### DETAILED DESCRIPTION OF THE INVENTION

### Compounds

The present invention is directed to 1,4-disubstituted piperidine, 1,4-disubstituted piperazine, 1,4-disubstituted diazepane, and 1,3-disubstituted pyrrolidine compounds. In particular, the present invention is directed to a compound of formula (I), without regard to chirality:

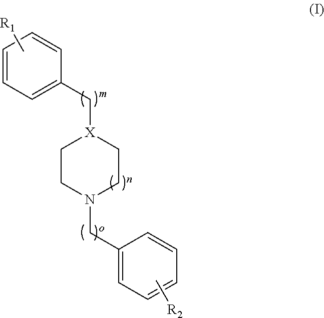

wherein

m is an integer in the range from 1 to 3;

n is zero or an integer in the range from 1 to 2;

o is an integer in the range from 1 to 3;

X represents either a nitrogen atom or a carbon atom with a single hydrogen attached; and

R₁ and R₂ are independently selected from the group consisting of hydrogen; methyl; deuteromethyl (CD₃); tritiomethyl (CT₃); ethyl; propyl; isopropyl; C₄-C₇ straight chain or branched alkyl; C₃-C₆ cycloalkyl; C₄-C₇ alkenyl (including cis and trans geometrical forms); benzyl; phenylethyl; amino; N-methylamino; N,N-dimethylamino; carboxylate; methylcarboxylate; ethylcarboxylate; propylcarboxylate; isopropylcarboxylate; carboxaldehyde; acetoxy; propionyloxy; isopropionyloxy; cyano; aminomethyl; N-methylaminomethyl; N,N-dimethylaminomethyl; carboxamide; N-methylcarboxamide; N,N-dimethylcarboxamide; acetyl; propionyl; formyl; benzoyl sulfate; phenyl; methyl sulfate; hydroxyl; methoxy; ethoxy; propoxy; isopropoxy; thiol; methylthio; ethylthio; propiothiol; fluoro; chloro; bromo; iodo; trifluoromethyl; vinyl; allyl; propargyl; nitro; carbamoyl; ureido; azido; isocyanate; thioisocyanate; hydroxylamino; nitroso; a saturated or unsaturated hydrocarbon ring; a nitrogen containing heterocyclic moiety; an oxygen containing heterocyclic moiety; a sulfur containing heterocyclic moiety; a selenium containing heterocyclic moiety; a mixed heterocyclic moiety containing at least two atoms selected from the group consisting of nitrogen, oxygen and sulfur; and ortho, meta or para-substituted benzene,

or a pharmaceutically acceptable salt thereof.

One or more R₁ is present in formula (I) and one or more R₂ is present in formula (I). In one embodiment, one R₁ is present in formula (I) and one R₂ is present in formula (I).

In one embodiment, R₁ and R₂ are independently selected from the group consisting of hydrogen, methyl, deuteromethyl (CD₃), tritiomethyl (CT₃), ethyl, propyl, isopropyl, C₄-C₇ straight chain or branched alkyl, C₃-C₆ cycloalkyl, C₄-C₇ alkenyl (including cis and trans geometrical forms), benzyl, phenylethyl, amino, N-methylamino, N,N-dimethylamino, carboxylate, methylcarboxylate, ethylcarboxylate, propylcarboxylate, isopropylcarboxylate, carboxaldehyde, acetoxy, propionyloxy, isopropionyloxy, cyano, acetyl, propionyl, formyl, phenyl, hydroxyl, methoxy, ethoxy, propoxy, isopropoxy, thiol, methylthio, ethylthio, propiothiol, fluoro, chloro, bromo, iodo, trifluoromethyl, vinyl, allyl, propargyl, nitro, azido, isocyanate, thioisocyanate, and nitroso.

In another embodiment, R₁ and R₂ are independently selected from the group consisting of hydrogen, methyl, ethyl, propyl, isopropyl, C₄-C₇ straight chain or branched alkyl, C₃-C₆ cycloalkyl, C₄-C₇ alkenyl (including cis and trans geometrical forms), benzyl, phenylethyl, carboxylate, methylcarboxylate, ethylcarboxylate, propylcarboxylate, isopropylcarboxylate, carboxaldehyde, cyano, acetyl, propionyl, formyl, phenyl, hydroxyl, methoxy, ethoxy, propoxy, isopropoxy, thiol, methylthio, ethylthio, propiothiol, fluoro, chloro, bromo, iodo, trifluoromethyl, vinyl, allyl, propargyl, nitro, azido, isocyanate, thioisocyanate, and nitroso.

In another embodiment, R₁ and R₂ are independently selected from the group consisting of hydrogen, methyl, ethyl, propyl, isopropyl, C₄-C₇ straight chain or branched alkyl, C₃-C₆ cycloalkyl, benzyl, cyano, hydroxyl, methoxy, ethoxy, propoxy, isopropoxy, thiol, methylthio, ethylthio, propiothiol, fluoro, chloro, bromo, iodo, trifluoromethyl, nitro, isocyanate, thioisocyanate, and nitroso.

In another embodiment, R₁ and R₂ are independently selected from the group consisting of hydrogen, methyl, ethyl, propyl, isopropyl, C₄-C₇ straight chain or branched alkyl, C₃-C₆ cycloalkyl, hydroxyl, methoxy, ethoxy, propoxy, isopropoxy, thiol, methylthio, ethylthio, propiothiol, fluoro, chloro, bromo, iodo, and trifluoromethyl.

In another embodiment, R₁ and R₂ are independently selected from the group consisting of hydrogen, methoxy, ethoxy, propoxy, isopropoxy, fluoro, chloro, bromo, and iodo.

In one embodiment, n is 1; X represents N or CH; and R₁ and R₂ are independently selected from the group consisting of hydrogen, methoxy, ethoxy, propoxy, isopropoxy, fluoro, chloro, bromo, and iodo.

In one embodiment, n is 2; X is N; and R₁ and R₂ are independently selected from the group consisting of hydrogen, methoxy, ethoxy, propoxy, isopropoxy, fluoro, chloro, bromo, and iodo.

Compounds of formula (I) include the following compounds:

1. m=2, n=1, o=2, X═CH, R₁═H, R₂=2-MeO

2. m=2, n=1, o=2, X═CH, R₁═H, R₂=3-MeO

3. m=2, n=1, o=2, X═CH, R₁═H, R₂=3,4-DiMeO

4. m=2, n=1, o=2, X═CH, R₁═H, R₂=4-MeO

5. m=1, n=1, o=2, X═CH, R₁═H, R₂=2-MeO

6. m=1, n=1, o=2, X═CH, R₁═H, R₂=3-MeO

7. m=1, n=1, o=2, X═CH, R₁═H, R₂=3,4-DiMeO

8. m=1, n=1, o=2, X═CH, R₁═H, R₂=4-MeO

9. m=2, n=1, o=2, X═CH, R₁=2,4,5=TriMeO, R₂═H

10. m=2, n=1, o=2, X═CH, R₁═H, R₂=2-Cl

11. m=2, n=1, o=2, X═CH, R₁═H, R₂=4-F

12. m=2, n=1, o=2, X═CH, R₁=2-MeO, R₂=2-MeO

13. m=2, n=1, o=2, X═CH, R₁=2-MeO, R₂═H

14. m=2, n=1, o=2, X═CH, R₁=3-MeO, R₂═H

15. m=2, n=1, o=2, X═CH, R₁=3-MeO, R₂=2-MeO

16. m=2, n=1, o=2, X═CH, R₁=3-MeO, R₂=3-MeO

17. m=2, n=1, o=2, X═CH, R₁=3=MeO, R₂=4-MeO

18. m=2, n=1, o=2, X═CH, R₁=3-MeO, R₂=2-Cl

19. m=2, n=1, o=2, X═CH, R₁=3-MeO, R₂=4-F

20. m=2, n=1, o=2, X═CH, R₁=4-MeO, R₂═H

21. m=2, n=1, o=2, X═CH, R₁=4-MeO, R₂=2-MeO

22. m=2, n=1, o=2, X═CH, R₁=4-MeO, R₂=3-MeO

23. m=2, n=1, o=2, X═CH, R₁=4-MeO, R₂=4-MeO

24. m=2, n=1, o=2, X═CH, R₁=4-MeO, R₂=2-Cl

25. m=2, n=1, o=2, X═CH, R₁=4-MeO, R₂=4-F

26. m=2, n=1, o=2, X═CH, R₁=2-MeO, R₂=3-MeO

27. m=2, n=1, o=2, X═CH, R₁=2-MeO, R₂=4-MeO

28. m=2, n=1, o=2, X═CH, R₁=2-MeO, R₂=2-Cl

29. m=2, n=1, o=2, X═CH, R₁=2-MeO, R₂=4-F

30. m=2, n=1, o=2, X═CH, R₁=2,4-DiF, R₂═H

31. m=2, n=1, o=2, X═CH, R₁=2,4-DiF, R₂=2-MeO

32. m=2, n=1, o=2, X═CH, R₁=2,4-DiF, R₂=3-MeO

33. m=2, n=1, o=2, X═CH, R₁=2,4-DiF, R₂=4-MeO

34. m=2, n=1, o=2, X═CH, R₁=2,4-DiF, R₂=2-Cl

35. m=2, n=1, o=2, X═CH, R₁=2,4-DiF, R₂=4-F

36. m=2, n=1, o=2, X═CH, R₁=2-F,4-MeO, R₂═H

37. m=2, n=1, o=2, X═CH, R₁=2-F,4-MeO, R₂=2-MeO

38. m=2, n=1, o=2, X═CH, R₁=2-F,4-MeO, R₂=3-MeO

39. m=2, n=1, o=2, X═CH, R₁=2-F,4-MeO, R₂=4-MeO

40. m=2, n=1, o=2, X═CH, R₁=2-F,4-MeO, R₂=2-Cl

41. m=2, n=1, o=2, X═CH, R₁=2-F,4-MeO, R₂=4-F

42. m=2, n=1, o=2, X═CH, R₁=3,5-DiF, R₂═H

43. m=2, n=1, o=2, X═CH, R₁=3,5-DiF, R₂=2-MeO

44. m=2, n=1, o=2, X═CH, R₁=3,5-DiF, R₂=3-MeO

45. m=2, n=1, o=2, X═CH, R₁=3,5-DiF, R₂=4-MeO

46. m=2, n=1, o=2, X═CH, R₁=3,5-DiF, R₂=2-Cl

47. m=2, n=1, o=2, X═CH, R₁=3,5-DiF, R₂=4-F

48. m=2, n=1, o=1, X═CH, R₁═H, R₂═H

49. m=2, n=1, o=1, X═CH, R₁═H, R₂=2-MeO

50. m=2, n=1, o=1, X═CH, R₁═H, R₂=3-MeO

51. m=2, n=1, o=1, X═CH, R₁═H, R₂=4-MeO

52. m=2, n=1, o=1, X═CH, R₁═H, R₂=2-Cl

53. m=2, n=1, o=1, X═CH, R₁═H, R₂=4-F

54. m=2, n=1, o=2, X═N, R₁═H, R₂═H

55. m=2, n=1, o=2, X═N, R₁=2-MeO, R₂=2-MeO

56. m=2, n=1, o=2, X═N, R₁=3-MeO, R₂=3-MeO

57. m=2, n=1, o=2, X═N, R₁=4-MeO, R₂=4-MeO

58. m=2, n=1, o=2, X═N, R₁=2-Cl, R₂=2-Cl

59. m=2, n=1, o=2, X═N, R₁=4-F, R₂=4-F

60. m=2, n=1, o=2, X═N, R₁═H, R₂=2-MeO

61. m=2, n=1, o=2, X═N, R₁═H, R₂=3-MeO

62. m=2, n=1, o=2, X═N, R₁═H, R₂=4-MeO

63. m=2, n=1, o=2, X═N, R₁═H, R₂=2-Cl

64. m=2, n=1, o=2, X═N, R₁═H, R₂=4-F

65. m=2, n=2, o=2, X═N, R₁═H, R₂═H

66. m=2, n=2, o=2, X═N, R₁=2-MeO, R₂=2-MeO

67. m=2, n=2, o=2, X═N, R₁=3-MeO, R₂=3-MeO

68. m=2, n=2, o=2, X═N, R₁=4-MeO, R₂=4-MeO

69. m=2, n=2, o=2, X═N, R₁=2-Cl, R₂=2-Cl

70. m=2, n=2, o=2, X═N, R₁=4-F, R₂=4-F

In one embodiment, m and o are 2; n is 1; and X is CH. In another embodiment, m and o are 2; n is 1; X is CH or N; R₁ is selected from the group consisting of H, 2-MeO, 2-F, and 4-MeO; and R₂ is selected from the group consisting of H, 4-F, 2-Cl, 2-MeO, and 3-MeO.

Compounds selected from the group consisting of 1-(2-methoxyphenethyl)-4-phenethylpiperidine, 1,4-diphenethylpiperazine, 1,4-bis(2-methoxyphenethyl)piperidine, -(3-methoxyphenethyl)-4-(2-methoxyphenethyl)piperidine, 1-(2-chlorophenethyl)-4-(2-methoxyphenethyl)piperidine, 1-(4-fluorophenethyl)-4-(2-methoxyphenethyl)piperidine, and 4-(2-fluoro-4-methoxyphenethyl)-1-(3-methoxyphenethyl)piperidine are included in the present invention.

In one embodiment, the compound of formula (I) is 1,4-bis(2-methoxyphenethyl)piperidine or a pharmaceutically acceptable salt thereof.

The 1,4-disubstituted piperidines, 1,4-disubstituted piperazines, 1,4-disubstituted diazepanes, and 1,3-disubstituted pyrrolidine compounds disclosed herein as well as analogs thereof include free base forms and salt forms, including soluble salt forms. Preferred salts include, for example, hydrochloride, hydrobromide, nitrate, sulfate, phosphate, tartrate, galactarate, fumarate, citrate, maleate, glycolate, malate, ascorbate, lactate, aspartate, glutamate, methanesulfonate, p-toluenesulfonate, benzenesulfonate, salicylate, proprionate, and succinate salts. The salt forms may be in some cases hydrates or solvates with alcohols and other solvents.

Compounds of the invention are synthesized according to methods known by one of ordinary skill in the art. See, for example, Bioorg Med Chem, 2005, 3899-3909.

In an embodiment, the compound of formula (I) contains aryl or heterocyclic moieties replacing the two phenyl moieties. One or more of the aryl and heterocyclic moieties may be substituted.

### Methods of Treatment

The invention further includes methods of treatment utilizing a compound of formula (I). Such methods of treatment include the following: a method of treating an eating disorder in an individual in need thereof, comprising administering to the individual a compound of formula (I) or a pharmaceutically acceptable salt thereof; a method of treating a disease or pathology of the central nervous system in an individual in need thereof, comprising administering to the individual a compound of formula (I) or a pharmaceutically acceptable salt thereof; and a method of treating an individual for drug dependence/abuse or withdrawal from drug dependence/abuse, comprising administering to the individual a compound of formula (I) or a pharmaceutically acceptable salt thereof.

In one embodiment, in the methods of treatment disclosed herein, the compound of formula (I) is administered in an effective amount being an amount of a drug effective to reduce an individual's desire for a drug of abuse or for alleviating at least one of the symptoms of the disease or pathological symptom of a central nervous system pathology.

In one embodiment, the drug is selected from the group consisting of cocaine, amphetamines, caffeine, nicotine, phencyclidine, opiates, barbiturates, benzodiazepanes, cannabinoids, hallucinogens, alcohol, and combinations thereof.

In one embodiment of the method of treating an individual for drug dependence, the method reduces the individual's desire for the drug of abuse by at least one day. Preferably, the method of treating an individual for drug dependence further comprises administering behavior modification counseling to the individual.

Although the compounds disclosed herein are contemplated primarily for the treatment of drug dependence/abuse and withdrawal from drug dependence/abuse, other uses are suggested by the studies discussed herein. For example, diseases and pathologies of the central nervous system that can be treated include cognitive disorders, brain trauma, memory loss, psychosis, sleep disorders, obsessive compulsive disorders, panic disorders, myasthenia gravis, Parkinson's disease, Alzheimer's disease, schizophrenia, Tourette's syndrome, Huntington's disease, attention deficit disorder, hyperkinetic syndrome, chronic nervous exhaustion, narcolepsy, pain, motion sickness, and depression.

### Pharmaceutical Composition

Also disclosed herein is a pharmaceutical composition comprising a compound of formula (I). For example, the pharmaceutical composition may include a conventional additive, such as a stabilizer, buffer, salt, preservative, filler, flavor enhancer and the like, as known to those skilled in the art. Representative buffers include phosphates, carbonates, and citrates. Exemplary preservatives include EDTA, EGTA, BHA, and BHT.

The pharmaceutical composition disclosed herein may be administered by inhalation (i.e., intranasally as an aerosol or nasal formulation), topically (i.e., in the form of an ointment, cream or lotion), orally (i.e., in solid or liquid form (tablet, gel cap, time release capsule, powder, solution, or suspension in aqueous or non-aqueous liquid), intravenously as an infusion or injection (i.e., as a solution, suspension or emulsion in a pharmaceutically acceptable carrier), transdermally (e.g., via a transdermal patch), or rectally as a suppository.

### Administration

The compounds disclosed herein can be administered alone, combined with an excipient, or co-administered with a second drug. Co-administration may provide a similar or synergistic effect. A compound of formula (I) or a pharmaceutically acceptable salt thereof can be administered subcutaneously, intramuscularly, intravenously, transdermally, orally, intranasally, intrapulmonary, or rectally.

Generally, the pharmacologically effective dose is in the amount ranging from about 1×10⁻⁵ to about 1 mg/kg body weight/day. The amount to be administered depends to some extent on the lipophilicity of the specific compound selected, since it is expected that this property of the compound will cause it to partition into fat deposits of the subject. The precise amount to be administered can be determined by the skilled practitioner in view of desired dosages, side effects and medical history of the patient and the like.

### EXAMPLES

### Example 1

### Compound 1. 1-(2-methoxyphenethyl)-4-phenethylpiperidine

A 250 mL round bottom flask was equipped with a magnetic stir bar, and then charged with 5 grams (0.0537 mol) of 4-picoline, 6.84 grams (0.0644 mol) of benzaldehyde, and 50 mL of acetic anhydride. The reaction mass was heated to reflux and maintained at that temperature for 72 hours. The reaction mixture was then cooled to room temperature, and subjected to silica chromatography. Yield of (E)-4-styrylpyridine was 5.2 grams (53%). The 5.2 grams of (E)-4-styrylpyridine was then charged into a 500 ml hydrogenation flask, to which was added 50 mL of acetic acid as well as 43 mg of PtO₂. The reaction mass was subjected to 45 psi of hydrogen gas, and allowed to react at room temperature for 16 hours. The reaction mixture was then filtered through a pad of celite, evaporated, basified with aqueous Na₂CO₃ solution, and extracted with dichloromethane. The combined extraction solvents were removed under reduced pressure via rotovap. The residue was then subjected to silica chromatography, yielding 4.6 grams (78.4% yield) of 4-phenethylpiperidine. A 100 mL round bottomed flask equipped with a magnetic stir bar was then charged with 2.0 grams of 4-phenethylpiperidine (0.0106 mol), 2.5 grams of 2-methoxyphenethylbromide (0.0127 mol), 3.65 grams of K₂CO₃ (0.0264 mol), and 25 mL of DMF as solvent. The reaction mass was then heated to 70° C. for 24 hours. The excess DMF was removed via reduced pressure; partitioned with water and dichloromethane; the organic layer separated; excess solvent removed under reduced pressure and the residue was subjected to silica chromatography. Yield of 1-(2-methoxyphenethyl)-4-phenethylpiperidine was 2.14 grams (62.3% yield). ¹H NMR (300 MHz, CDCl₃) δ 1.31-1.63 (m, 7H), 2.32-2.85 (in, 1.0H), 3.87 (s, 3H), 6.83-7.35 (m, 9H) ppm.

### Example 2

### Compound 54. 1,4-diphenethylpiperazine

A 250 mL round bottomed flask equipped with a magnetic stir bar was then charged with 2.0 grams (0.0232 mol) of 1,4-piperazine, 11.16 grams (0.06 mol) of phenethylbromide, 16 grams (0.12 mol) of K₂CO₃; and 100 mL of DMF as solvent. The reaction mass was then heated to 70° C. for 24 hours. The excess DMF was removed via reduced pressure, partitioned with water and dichloromethane, the organic layer separated; excess solvent removed under reduced pressure and the residue was subjected to silica chromatography. Yield of 1,4-diphenethylpiperazine was 3.7 grains (54.7% yield). NMR (300 MHz, CDCl₃) δ 2.45-2.87 (m, 16H), 7.18-7.35 (m, 10H) ppm.

### Example 3

### Compound 12. 1,4-bis(2-methoxyphenethyl)piperidine

A 250 mL round bottom flask was equipped with a magnetic stir bar, and then charged with 5 grams (0.0537 mol) of 4-picoline, 8.77 grams (0.0644 mol) of 2-methoxybenzaldehyde, and 50 mL of acetic anhydride. The reaction mass was heated to reflux and maintained at that temperature for 72 hours. The reaction mixture was then cooled to room temperature, and subjected to silica chromatography. Yield of (E)-4-(2-methoxystyryl)pyridine was 7.15 grams (63%). The 7.15 grams of (E)-4-(2-methoxystyryl)pyridine was then charged into a 500 ml hydrogenation flask, to which was added 100 mL of acetic acid as well as 50 mg of PtO₂. The reaction mass was subjected to 45 psi of hydrogen gas, and allowed to react at room temperature for 16 hours. The reaction mixture was then basified, and extracted with dichloromethane, and excess extraction solvent was removed under reduced pressure via rotovap. The residue was then subjected to silica chromatography, yielding 6.1 grams (82.1% yield) of 4-(2-methoxyphenethyl)piperidine. A 100 mL round bottom flask equipped with a magnetic stir bar was then charged with 1.0 grams of 4-(2-methoxyphenethyl)piperidine (0.0046 mol), 1.25 grams of 2-methoxyphenethylbromide (0.0064 mol), 1.87 grams of K₂CO₃ (0.0135 mol), and 20 mL of DMF as solvent. The reaction mass was then heated to 70° C. for 24 hours. The excess DMF was removed via reduced pressure, extracted with water and dichloromethane, the organic layer separated; excess solvent removed under reduced pressure and the residue was subjected to silica chromatography. Yield of 1,4-bis(2-methoxyphenethyl)piperidine was 1.04 grams (64.0% yield). ¹H NMR (300 MHz, CDCl₃) δ 1.33-1.59 (m, 7H), 2.35-2.80 (m, 10H), 3.82 (s, 6H), 6.83-7.21 (m, 8H) ppm.

### Example 4

### Compound 26. 1-(3-methoxyphenethyl)-4-(2-methoxyphenethyl)piperidine

A 250 mL round bottom flask was equipped with a magnetic stir bar, and then charged with 5 grams (0.0537 mol) of 4-picoline, 8.77 grams (0.0644 mol) of 2-methoxybenzaldehyde, and 50 mL of acetic anhydride. The reaction mass was heated to reflux and maintained at that temperature for 72 hours. The reaction mixture was then cooled to room temperature, and subjected to silica chromatography. Yield of (E)-4-(2-methoxystyryl)pyridine was 7.15 grams (63%). The 7.15 grams of (E)-4-(2-methoxystyryl)pyridine was then charged into a 500 mL hydrogenation flask, to which was added 100 mL of acetic acid as well as 50 mg of PtO₂. The reaction mass was subjected to 45 psi of hydrogen gas, and allowed to react at room temperature for 16 hours. The reaction mixture was then filtered through a pad of celite, evaporated, basified with aqueous Na₂CO₃ solution, and extracted with dichloromethane. The combined extraction solvents were removed under reduced pressure via rotovap. The residue was then subjected to silica chromatography, yielding 6.1 grams (82.1% yield) of 4-(2-methoxyphenethyl)piperidine. A 100 mL round bottomed flask equipped with a magnetic stir bar was then charged with 1.0 grams of 4-(2-methoxyphenethyl)piperidine (0.0046 mol), 1.25 grams of 3-methoxyphenethylbromide (0.0064 mol), 1.87 grams of K₂CO₃ (0.0135 mol), and 20 mL of DMF as solvent. The reaction mass was then heated to 70° C. for 24 hours. The excess DMF was removed via reduced pressure, partitioned with water and dichloromethane, the organic layer separated; excess solvent removed under reduced pressure and the residue was subjected to silica chromatography. Yield of 1-(3-methoxyphenethyl)-4-(2-methoxyphenethyl)piperidine was 0.93 grains (57.1% yield). ¹H NMR (300 MHz, CDCl₃) δ 1.31-1.55 (m, 7H), 2.35-2.65 (m, 10H), 3.81 (s, 6H), 6.80-7.37 (m, 8H) ppm.

### Example 5

### Compound 28. 1-(2-chlorophenethyl)-4-(2-methoxyphenethyl)piperidine

A 250 mL round bottom flask was equipped with a magnetic stir bar, and then charged with 5 grams (0.0537 mol) of 4-picoline, 8.77 grams (0.0644 mol) of 2-methoxybenzaldehyde, and 50 ml of acetic anhydride. The reaction mass was heated to reflux and maintained at that temperature for 72 hours. The reaction mixture was then cooled to room temperature, and subjected to silica chromatography. Yield of (E)-4-(2-methoxystyryl)pyridine was 7.15 grams (63%). The 7.15 grams of (E)-4-(2-methoxystyryl)pyridine was then charged into a 500 mL hydrogenation flask, to which was added 100 mL of acetic acid as well as 50 mg of PtO₂. The reaction mass was subjected to 45 psi of hydrogen gas, and allowed to react at room temperature for 16 hours. The reaction mixture was then filtered through a pad of celite, evaporated, basified with aqueous Na₂CO₃ solution, and extracted with dichloromethane. The combined extraction solvents were removed under reduced pressure via rotovap. The residue was then subjected to silica chromatography, yielding 6.1 grams (82.1% yield) of 4-(2-methoxyphenethyl)piperidine. A 100 ml, round bottom flask equipped with a magnetic stir bar was then charged with 1.0 grams of 4-(2-methoxyphenethyl)piperidine (0.0046 mol), 1.41 grams of 2-chlorophenethyllbromide (0.0064 mol), 1.87 grams of K₂CO₃ (0.0135 mol), and 20 mL of MIT as solvent. The reaction mass was then heated to 70° C. for 24 hours. The excess MIT was removed via reduced pressure, partitioned with water and dichloromethane, the organic layer separated; excess solvent removed under reduced pressure and the residue was subjected to silica chromatography. Yield of 1-(2-chlorophenethyl)-4-(2-methoxyphenethyl)piperidine was 1.12 grams (68.0% yield). ¹H NMR (300 MHz, CDCl₃) δ 1.40-1.62 (m, 7H), 2.41-2.75 (m, 10H), 3.84 (s, 3H), 6.92-7.73 (m, 8H) ppm.

### Example 6

### Compound 29. 1-(4-fluorophenethyl)-4-(2-methoxyphenethyl)piperidine

A 250 mL round bottom flask was equipped with a magnetic stir bar, and then charged with 5 grams (0.0537 mol) of 4-picoline, 8.77 grams (0.0644 mol) of 2-methoxybenzaldehyde, and 50 ml of acetic anhydride. The reaction mass was heated to reflux and maintained at that temperature for 72 hours. The reaction mixture was then cooled to room temperature, and subjected to silica chromatography. Yield of (E)-4-(2-methoxystyryl)pyridine was 7.15 grams (63%). The 7.15 grams of (E)-4-(2-methoxystyryl)pyridine was then charged into a 500 mL hydrogenation flask, to which was added 100 mL of acetic acid as well as 50 mg of PtO₂. The reaction mass was subjected to 45 psi of hydrogen gas, and allowed to react at room temperature for 16 hours. The reaction mixture was then filtered through a pad of celite, evaporated, basified with aqueous Na₂CO₃ solution, and the combined extraction solvents were removed under reduced pressure via rotovap. The residue was then subjected to silica chromatography, yielding 6.1 grams (82.1% yield) of 4-(2-methoxyphenethyl)piperidine. A 100 mL round bottom flask equipped with a magnetic stir bar was then charged with 1.0 grams of 4-(2-methoxyphenethyl)piperidine (0.0046 mol), 1.3 grams of 4-fluorophenethylbromide (0.0064 mol), 1.87 grains of K₂CO₃ (0.0135 mol), and 20 mL of DMF as solvent. The reaction mass was then heated to 70° C. for 24 hours. The excess DMF was removed via reduced pressure, partitioned with water and dichloromethane, the organic layer separated; excess solvent removed under reduced pressure and the residue was subjected to silica chromatography. Yield of 1-(4-fluorophenethyl)-4-(2-methoxyphenethyl)piperidine was 0.99 grams (63.0% yield). ¹H NMR (300 MHz, CDCl₃) δ 1.33-1.55 (m, 7H), 2.33-2.60 (m, 10H), 3.80 (s, 3H), 6.80-7.32 (in, 8H) ppm.

### Example 7

### [³H]Dihydrotetrabenazine ([³H]DTBZ) Binding Assay, Vesicular Preparation

Synaptic vesicles were prepared from rat brain using a modification of a previously described procedure (Teng et al., 1998). Briefly, fresh whole brain (excluding cerebellum) was homogenized using a Teflon pestle (clearance 0.003 inches) with 7 vertical strokes at 800 rpm in 20 vol of ice-cold 0.32 M sucrose and centrifuged at 1000 g for 12 min at 4° C. The resulting supernatant (S₁) was then centrifuged at 22,000 g for 10 min at 4° C. The synaptosomal pellets (P₂) were homogenized in 18 mL of ice-cold Milli-Q water and exposed for 5 min for lysing synaptosomes. Osmolarity was restored by addition of 2 mL of 25 mM HEPES with 100 mM dipotassium tartrate (pH 7.5). Samples were centrifuged at 20,000 g for 20 min at 4° C. to remove lysed synaptosomal membranes. MgSO₄ (1 mM) was added to the supernatant (S₃), and was centrifuged at 100,000 g for 45 min at 4° C. The final vesicular pellets (P₄) were resuspended in ice-cold assay buffer (see below) providing ˜15 μg protein/100 μL, determined by the method of Bradford (1976) using bovine serum albumin as a the standard. Aliquot parts (100 μL) of suspension of vesicle membrane protein were incubated in assay buffer (25 mM HEPES, 100 mM dipotassium tartrate, 5 mM MgSO₄, 0.1 mM EDTA and 0.05 mM EGTA, pH 7.5, at 25° C.) in the presence of 3 nM [³H]DTBZ and at least 7 concentrations (1 nM-1 mM) of compound for 1 hr at room temperature. Nonspecific binding was determined in the presence of 20 μM tetrabenazine, a standard compound. Assays were performed in duplicate using a 96-well plate format. Reactions were terminated by filtration of samples on a Unifilter-96 GF/B filter plates (presoaked in 0.5% polyethylenimine), using a FilterMate harvester (Packard BioScience Co., Meriden, Conn.).

After washing 5 times with 350 μL of the ice-cold wash buffer (25 mM HEPES, 100 mM dipotassium tartrate, 5 mM MgSO₄ and 10 mM NaCl, pH 7.5), filter plates were dried, sealed and each well filled with 40 μL Packard's MicroScint 20 cocktail. Bound [³H]DTBZ was measured using a Packard TopCount NXT scintillation counter with a Packard Windows NT based operating system.

### Example 8

### [³H]Dopamine ([³H]DA) Uptake Assay, Vesicular Preparation

Inhibition of [³H]DA uptake was conducted using isolated synaptic vesicle preparations (Teng et al., 1997). Briefly, rat striata were homogenized with 10 up-and-down strokes of a Teflon pestle homogenizer (clearance ˜0.003″) in 14 ml of 0.32 M sucrose solution. Homogenates were centrifuged (2,000 g for 10 min at 4° C.), and then the supernatants were centrifuged (10,000 g for 30 min at 4° C.). Pellets were resuspended in 2 ml of 0.32 M sucrose solution and subjected to osmotic shock by adding 7 ml of ice-cold MilliQ water to the preparation. After 1 min, osmolarity was restored by adding 900 μl of 0.25 M HEPES buffer and 900 μl of 1.0 M potassium tartrate solution. Samples were centrifuged (20,000 g for 20 min at 4° C.), and the supernatants were centrifuged (55,000 g for 1 hr at 4° C.), followed by addition of 100 μl of 10 mM MgSO₄, 100 μl of 0.25 M HEPES and 100 μl of 1.0 M potassium tartrate solution prior to the final centrifugation (100,000 g for 45 min at 4° C.). Final pellets were resuspended in 2.4 ml of assay buffer (25 mM HEPES, 100 mM potassium tartrate, 50 μM EGTA, 100 μM EDTA, 1.7 mM ascorbic acid, 2 mM ATP-Mg²⁺, pH 7.4). Aliquots of the vesicular suspension (100 μl) were added to tubes containing assay buffer, various concentrations of compound (0.1 nM-10 mM) and 0.1 μM [³H]DA in a final volume of 500 and incubated at 37° C. for 8 min. Nonspecific uptake was determined in the presence of the standard compound, Ro4-1284 (10 μM). Reactions were terminated by filtration, and radioactivity retained by the filters was determined by liquid scintillation spectrometry (Tri-Carb 2100TR liquid scintillation analyzer; PerkinElmer Life and Analytical Sciences, Boston, Mass.).

### Example 9

### [³H]Dofetilide Binding Assay, HEK-293 Cell Membrane Preparation

[³H]Dofetilide binding assays were conducted using commercially available HEK-293 cell membranes which stably express the hERG channel. Membranes were suspended in assay buffer (50 mM Tris, 10 mM KCl, 1 mM MgCl₂, pH 7.4) prior to the experiment. Assays were performed in duplicate in a total volume of 250 μL. Aliquots of the HEK-293 cell membrane suspension which contained 5 μg membrane protein were added to tubes containing assay buffer, 5 nM [³H]dofetilide and a range of concentrations of analog (10 nM-100 μM). Nonspecific binding was determined in the presence of amitriptyline (1 mM). Samples were incubated for 1 hr. at 24° C., followed by rapid filtration. Radioactivity retained by the filters was determined by liquid scintillation spectrometry as described above for the [³H]DA uptake assay. The affinity for the [³H]dofetilide binding site on the hERG channel expressed in the HEK-293 cellular membrane was determined from the analog concentration response curves.

### Example 10

### [³H]DA and [³H]5-HT Uptake Assay, Synaptosomal Preparation

[³H]DA and [³H]5-HT uptake into striatal synaptosomes was determined to evaluate compound inhibition of the dopamine transporter (DAT) and the serotonin transporter (SERT), respectively. Striata from individual rats were homogenized in ice-cold sucrose solution containing 5 mM NaHCO₃ (pH 7.4), with 16 up-and-down strokes of a Teflon pestle homogenizer (clearance≈0.003″). Homogenates were centrifuged at 2000 g for 10 min at 4° C., and resulting supernatants were centrifuged at 20,000 g for 17 min at 4° C. Pellets were resuspended in 2.4 mL (for DAT assays) or 1.5 mL (for SERT assays) of assay buffer (125 mM NaCl, 5 mM KCl, 1.5 mM MgSO₄, 1.25 mM CaCl₂, 1.5 mM KH₂PO₄, 10 mM alpha-D-glucose, 25 mM HEPES, 0.1 mM EDTA, 0.1 mM pargyline, 0.1 mM ascorbic acid, saturated with 95% 0₂/5% CO₂, pH 7.4). Assays were performed in duplicate in a total volume of 500 (for DAT assays) or 250 μL (for SERT assays). Aliquots of the synaptosomal suspension (25 μL for DAT, 50 μL for SERT) were added to tubes containing assay buffer and various concentrations of analog (1 nM-100 and incubated at 34° C. for 5 min. Nonspecific uptake was determined in the presence of nomifensine (10 μM) for DAT assays or fluoxetine (10 μM) for SERT assays. GBR-12935 (100 nM) was included in the assay buffer for the SERT assay to maximally inhibit [³H]5-HT uptake through DAT and isolate uptake to SERT. Samples were placed on ice, and 50 μL of 0.1 μM [³H]DA (for DAT assays) or 25 μL of 0.1 [³H]5-HT (for SERT assays) was added to each tube, and incubated for 10 min at 34° C. Reactions were terminated by addition of 3 mL of ice-cold assay buffer and subsequent filtration and radioactivity retained by the filters was determined by liquid scintillation spectrometry (Tri-Carb 2100TR liquid scintillation analyzer; PerkinElmer Life and Analytical Sciences, Boston, Mass.).

Exemplary compounds 1-70 were tested in [³H]Dihydrotetrabenazine ([³H]DTBZ) binding assay according to Example 7 and the [³H]Dopamine ([³H]DA) uptake assay according to Example 8. The results of these assays are set forth in Table 1.

TABLE 1

| | | | | | | | Inhibition of | [³H]DA |
|---|---|---|---|---|---|---|---|---|
| | | | | | | | [³H]DTBZ | Uptake (Ki) |
| | | | | | | | binding (Ki) | VMAT2 |
| Compound | m | n | o | X | R₁ | R₂ | VMAT2 (μM) | (μM) |
| 1 | 2 | 1 | 2 | CH | H | 2-MeO | 0.255 ± 0.026 | 0.030 ± 0.0020 |
| 2 | 2 | 1 | 2 | CH | H | 3-MeO | 0.265 ± 0.038 | 0.022 ± 0.00087 |
| 3 | 2 | 1 | 2 | CH | H | 3,4-DiMeO | 2.8 ± 0.11 | 0.056 ± 0.009 |
| 4 | 2 | 1 | 2 | CH | H | 4-MeO | 1.62 ± 0.19 | 0.046 ± 0.0037 |
| 5 | 1 | 1 | 2 | CH | H | 2-MeO | 2.76 ± 0.31 | 0.13 ± 0.015 |
| 6 | 1 | 1 | 2 | CH | H | 3-MeO | 1.53 ± 0.10 | 0.077 ± 0.0098 |
| 7 | 1 | 1 | 2 | CH | H | 3,4-DiMeO | 2.99 ± 0.17 | 0.24 ± 0.034 |
| 8 | 1 | 1 | 2 | CH | H | 4-MeO | 1.94 ± 0.43 | 0.18 ± 0.011 |
| 9 | 2 | 1 | 2 | CH | 2,4,5- | H | 0.38 ± 0.049 | 0.094 ± 0.0096 |
| | | | | | TriMeO | | | |
| 10 | 2 | 1 | 2 | CH | H | 2-Cl | 0.38 ± 0.0058 | 0.047 ± 0.0063 |
| 11 | 2 | 1 | 2 | CH | H | 4-F | 1.82 ± 0.079 | 0.032 ± 0.0038 |
| 12 | 2 | 1 | 2 | CH | 2-MeO | 2-MeO | 0.19 ± 0.0088 | 0.0093 ± 0.0006 |
| 13 | 2 | 1 | 2 | CH | 2-MeO | H | 0.69 ± 0.050 | 0.022 ± 0.0028 |
| 14 | 2 | 1 | 2 | CH | 3-MeO | H | 0.32 ± 0.015 | 0.056 ± 0.0086 |
| 15 | 2 | 1 | 2 | CH | 3-MeO | 2-MeO | 0.32 ± 0.055 | 0.040 ± 0.0075 |
| 16 | 2 | 1 | 2 | CH | 3-MeO | 3-MeO | 0.23 ± 0.019 | 0.085 ± 0.010 |
| 17 | 2 | 1 | 2 | CH | 3-MeO | 4-MeO | 0.45 ± 0.13 | 0.13 ± 0.023 |
| 18 | 2 | 1 | 2 | CH | 3-MeO | 2-Cl | 0.46 ± 0.098 | 0.42 ± 0.049 |
| 19 | 2 | 1 | 2 | CH | 3-MeO | 4-F | 0.96 ± 0.047 | 0.069 ± 0.004 |
| 20 | 2 | 1 | 2 | CH | 4-MeO | H | 0.51 ± 0.087 | 0.11 ± 0.007 |
| 21 | 2 | 1 | 2 | CH | 4-MeO | 2-MeO | 0.42 ± 0.019 | 0.083 ± 0.009 |
| 22 | 2 | 1 | 2 | CH | 4-MeO | 3-MeO | 0.23 ± 0.047 | 0.075 ± 0.004 |
| 23 | 2 | 1 | 2 | CH | 4-MeO | 4-MeO | 2.47 ± 0.27 | 0.16 ± 0.012 |
| 24 | 2 | 1 | 2 | CH | 4-MeO | 2-Cl | 0.23 ± 0.020 | 0.040 ± 0.007 |
| 25 | 2 | 1 | 2 | CH | 4-MeO | 4-F | 0.47 ± 0.067 | 0.060 ± 0.005 |
| 26 | 2 | 1 | 2 | CH | 2-MeO | 3-MeO | 0.15 ± 0.0058 | 0.013 ± 0.0015 |
| 27 | 2 | 1 | 2 | CH | 2-MeO | 4-MeO | 0.50 ± 0.10 | 0.043 ± 0.0028 |
| 28 | 2 | 1 | 2 | CH | 2-MeO | 2-Cl | 0.19 ± 0.020 | 0.020 ± 0.0030 |
| 29 | 2 | 1 | 2 | CH | 2-MeO | 4-F | 0.40 ± 0.097 | 0.013 ± 0.0021 |
| 30 | 2 | 1 | 2 | CH | 2,4-DiF | H | 0.53 ± 0.11 | 0.053 ± 0.0055 |
| 31 | 2 | 1 | 2 | CH | 2,4-DiF | 2-MeO | 0.35 ± 0.048 | 0.041 ± 0.012 |
| 32 | 2 | 1 | 2 | CH | 2,4-DiF | 3-MeO | 0.27 ± 0.015 | 0.029 ± 0.0055 |
| 33 | 2 | 1 | 2 | CH | 2,4-DiF | 4-MeO | 1.30 ± 0.17 | 0.070 ± 0.0051 |
| 34 | 2 | 1 | 2 | CH | 2,4-DiF | 2-Cl | 0.24 ± 0.038 | 0.11 ± 0.019 |
| 35 | 2 | 1 | 2 | CH | 2,4-DiF | 4-F | 1.45 ± 0.24 | 0.043 ± 0.014 |
| 36 | 2 | 1 | 2 | CH | 2-F, 4- | H | 0.29 ± 0.064 | 0.12 ± 0.0085 |
| | | | | | MeO | | | |
| 37 | 2 | 1 | 2 | CH | 2-F, 4- | 2-MeO | 0.33 ± 0.018 | 0.076 ± 0.010 |
| | | | | | MeO | | | |
| 38 | 2 | 1 | 2 | CH | 2-F, 4- | 3-MeO | 0.19 ± 0.009 | 0.083 ± 0.0038 |
| | | | | | MeO | | | |
| 39 | 2 | 1 | 2 | CH | 2-F, 4- | 4-MeO | 1.61 ± 0.078 | 0.044 ± 0.0033 |
| | | | | | MeO | | | |
| 40 | 2 | 1 | 2 | CH | 2-F, 4- | 2-Cl | 0.23 ± 0.015 | 0.067 ± 0.021 |
| | | | | | MeO | | | |
| 41 | 2 | 1 | 2 | CH | 2-F, 4- | 4-F | 0.60 ± 0.075 | 0.060 ± 0.0082 |
| | | | | | MeO | | | |
| 42 | 2 | 1 | 2 | CH | 3,5-DiF | H | 1.10 ± 0.087 | 0.051 ± 0.0035 |
| 43 | 2 | 1 | 2 | CH | 3,5-DiF | 2-MeO | 0.41 ± 0.038 | 0.034 ± 0.0039 |
| 44 | 2 | 1 | 2 | CH | 3,5-DiF | 3-MeO | 0.25 ± 0.003 | 0.028 ± 0.0027 |
| 45 | 2 | 1 | 2 | CH | 3,5-DiF | 4-MeO | 2.03 ± 0.13 | 0.12 ± 0.028 |
| 46 | 2 | 1 | 2 | CH | 3,5-DiF | 2-Cl | 0.66 ± 0.058 | 0.18 ± 0.068 |
| 47 | 2 | 1 | 2 | CH | 3,5-DiF | 4-F | 1.43 ± 0.28 | 0.068 ± 0.016 |
| 48 | 2 | 1 | 1 | CH | H | H | 21.7 ± 2.08 | 0.19 ± 0.063 |
| 49 | 2 | 1 | 1 | CH | H | 2-MeO | 9.36 ± 1.32 | 0.070 ± 0.0075 |
| 50 | 2 | 1 | 1 | CH | H | 3-MeO | 12.7 ± 4.28 | 0.19 ± 0.020 |
| 51 | 2 | 1 | 1 | CH | H | 4-MeO | 6.23 ± 0.56 | 0.25 ± 0.027 |
| 52 | 2 | 1 | 1 | CH | H | 2-Cl | 44.5 ± 17.8 | 0.27 ± 0.042 |
| 53 | 2 | 1 | 1 | CH | H | 4-F | 10.1 ± 0.58 | 0.21 ± 0.012 |
| 54 | 2 | 1 | 2 | N | H | H | 3.05 ± 0.64 | 0.11 ± 0.012 |
| 55 | 2 | 1 | 2 | N | 2-MeO | 2-MeO | 1.17 ± 0.13 | 0.035 ± 0.0012 |
| 56 | 2 | 1 | 2 | N | 3-MeO | 3-MeO | 1.05 ± 0.087 | 0.060 ± 0.004 |
| 57 | 2 | 1 | 2 | N | 4-MeO | 4-MeO | 6.63 ± 0.84 | 0.41 ± 0.005 |
| 58 | 2 | 1 | 2 | N | 2-Cl | 2-Cl | 0.37 ± 0.038 | 0.048 ± 0.003 |
| 59 | 2 | 1 | 2 | N | 4-F | 4-F | 3.71 ± 0.48 | 0.058 ± 0.012 |
| 60 | 2 | 1 | 2 | N | H | 2-MeO | 1.07 ± 0.25 | 0.037 ± 0.0005 |
| 61 | 2 | 1 | 2 | N | H | 3-MeO | 1.30 ± 0.33 | 0.098 ± 0.016 |
| 62 | 2 | 1 | 2 | N | H | 4-MeO | 4.63 ± 1.31 | 0.10 ± 0.011 |
| 63 | 2 | 1 | 2 | N | H | 2-Cl | 1.59 ± 0.26 | 0.063 ± 0.0069 |
| 64 | 2 | 1 | 2 | N | H | 4-F | 3.86 ± 1.01 | 0.088 ± 0.0069 |
| 65 | 2 | 2 | 2 | N | H | H | 3.17 ± 0.55 | 0.19 ± 0.017 |
| 66 | 2 | 2 | 2 | N | 2-MeO | 2-MeO | 2.02 ± 0.11 | 0.19 ± 0.026 |
| 67 | 2 | 2 | 2 | N | 3-MeO | 3-MeO | 2.24 ± 0.53 | 0.10 ± 0.010 |
| 68 | 2 | 2 | 2 | N | 4-MeO | 4-MeO | 2.88 ± 0.25 | 0.25 ± 0.015 |
| 69 | 2 | 2 | 2 | N | 2-Cl | 2-Cl | 0.93 ± 0.021 | 0.17 ± 0.025 |
| 70 | 2 | 2 | 2 | N | 4-F | 4-F | 5.05 ± 0.77 | 0.085 ± 0.0052 |

As shown in Table 1, exemplary compounds 1-70 exhibited activity at the vesicular monoamine transporter-2. Four of these compounds (compounds 12, 26, 28, 38) exhibited inhibition of [³H]DTBZ binding with Ki values ranging from 0.15-0.19 μM. A number of these compounds exhibited inhibition of [³H]DTBZ binding with Ki values ranging from 0.20-0.50 μM. Accordingly, the results in Table 1 demonstrate the compounds of formula (I) are effective in inhibiting the binding of [³H]DTBZ to vesicle membranes indicating an interaction with vesicular monoamine transporter-2

As shown in Table 1, exemplary compounds 1-70 exhibited activity at the vesicular monoamine transporter by inhibiting the uptake of dopamine into synaptic vesicle preparations. Four of these compounds (compounds 12, 26, 28, 29) exhibited inhibition of [³H]DA uptake with Ki values ranging from 9-20 nM. A number of these compounds exhibited inhibition of [³H]DA uptake with Ki values ranging from 20-50 nM. Accordingly, the results in Table 1 also demonstrate the compounds of formula (I) are effective in inhibiting uptake of extracellular dopamine by the cells of the central nervous system.

The results in Table 1 demonstrate the usefulness of the compounds of formula (I) in the methods of treatment disclosed herein.

Certain of the exemplary compounds were tested in the [³H]Dofetilide binding assay according to Example 9 and the [³H]DA and [³H]5-HT uptake assay according to Example 10. The results of these assays are set forth in Table 2.

In particular, to evaluate off-target interactions and to eliminate exemplary compounds that were not promising, the exemplary compounds which exhibited a Ki of <100 nM for inhibition of [³H]DA uptake at VMAT2 (the pharmacological target) were evaluated for their interaction with the human ether-a-go-go-related gene (hERG) channel in the [³H]Dofetilide binding assay according to Example 9. The hERG channel is an inward rectifying K⁺ channel in the heart. Accordingly, this test assessed the potential of the tested compounds to produce cardiac arrhythmias via interaction at this off-target site. The criterion for compounds to be considered leads was a 30-fold selectivity for VMAT2 over hERG.

Compounds meeting this criterion were further evaluated in the [³H]DA and [³H]5-HT uptake assay according to Example 10 to determine inhibition of [³H]DA and [³H]5-HT uptake, respectively, using striatal synaptosomal preparations. Compounds having high potency in inhibiting [³H]DA uptake at the dopamine transporter (DAT) and the serotonin transporter (SERT) have the potential for abuse liability. As such, the criterion of 30-fold greater selectivity for VMAT2 over DAT and SERT was required for lead compound status. Based on the results thus far, six out of the seventy compounds evaluated have successfully met the criteria for lead compound status: 12, 13, 24, 60, 61 and 62.

TABLE 2

| | | DAT [³H]DA Uptake | |
|---|---|---|---|
| | hERG [³H]Dofetilide | Inhibition | SERT [³H]5-HT Uptake |
| | Binding Inhibition | Mean ± SEM | Inhibition |
| Compound | Mean ± SEM (IC50; μM) | (Ki; μM) | Mean ± SEM (Ki; μM) |
| 1 | 0.534 ± 0.142 | N/A | N/A |
| 3 | 0.402 ± 0.0727 | N/A | N/A |
| 2 | 0.489 ± 0.0847 | 3.13 ± 0.19 | 1.15 ± 0.09 |
| 4 | 0.622 ± 0.215 | N/A | N/A |
| 5 | N/A | N/A | N/A |
| 6 | 0.442 ± 0.221 | N/A | N/A |
| 7 | N/A | N/A | N/A |
| 8 | N/A | N/A | N/A |
| 9 | 8.10 ± 0.585 | 5.46 ± 0.120 | N/A |
| 10 | 0.666 ± 0.0265 | N/A | N/A |
| 11 | 0.353 ± 0.0360 | N/A | N/A |
| 12 | 1.07 ± 0.0906 | 3.45 ± 0.45 | 0.12 ± 0.01 |
| 13 | 1.65 ± 0.670 | 3.56 ± 1.21 | 0.75 ± 0.18 |
| 14 | 0.631 ± 0.0888 | N/A | N/A |
| 15 | 0.790 ± 0.0641 | N/A | N/A |
| 16 | 0.564 ± 0.131 | N/A | N/A |
| 17 | 0.600 ± 0.0878 | N/A | N/A |
| 18 | N/A | N/A | N/A |
| 19 | 0.409 ± 0.0429 | N/A | N/A |
| 20 | N/A | N/A | N/A |
| 21 | 0.323 ± 0.0964 | N/A | N/A |
| 22 | 0.479 ± 0.228 | N/A | N/A |
| 23 | N/A | N/A | N/A |
| 24 | 2.09 ± 0.391 | 5.16 ± 1.25 | 4.15 ± 1.45 |
| 25 | 1.42 ± 0.384 | N/A | N/A |
| 26 | 0.607 ± 0.0255 | N/A | N/A |
| 27 | 0.479 ± 0.109 | N/A | N/A |
| 28 | 1.19 ± 0.181 | 1.25 ± 0.307 | N/A |
| 29 | 0.347 ± 0.0188 | 4.13 ± 0.44 | 0.58 ± 0.07 |
| 30 | 0.552 ± 0.0991 | N/A | N/A |
| 31 | 0.317 ± 0.0248 | N/A | N/A |
| 32 | 0.327 ± 0.0353 | N/A | N/A |
| 33 | 1.07 ± 0.152 | N/A | N/A |
| 34 | N/A | N/A | N/A |
| 35 | 0.201 ± 0.0327 | N/A | N/A |
| 36 | N/A | N/A | N/A |
| 37 | 2.87 ± 0.760 | N/A | N/A |
| 38 | 0.479 ± 0.0643 | 2.94 ± 0.270 | N/A |
| 39 | 0.299 ± 0.0459 | N/A | N/A |
| 40 | 1.41 ± 0.166 | N/A | N/A |
| 41 | 3.60 ± 1.22 | 3.48 ± 0.415 | N/A |
| 42 | 0.258 | N/A | N/A |
| 43 | 0.427 ± 0.134 | N/A | N/A |
| 44 | 0.477 ± 0.0429 | 1.91 ± 0.568 | N/A |
| 45 | N/A | N/A | N/A |
| 46 | N/A | N/A | N/A |
| 47 | 0.440 ± 0.04117 | N/A | N/A |
| 48 | N/A | N/A | N/A |
| 49 | 0.568 ± 0.122 | N/A | N/A |
| 50 | N/A | N/A | N/A |
| 51 | N/A | N/A | N/A |
| 52 | N/A | N/A | N/A |
| 53 | N/A | N/A | N/A |
| 54 | 3.39 ± 1.94 | 13.6 ± 2.69 | 11.4 ± 4.14 |
| 55 | 1.02 ± 0.180 | 14.4 ± 0.456 | 1.58 ± 0.415 |
| 56 | 1.49 ± 0.204 | 7.90 ± 2.89 | 3.62 ± 1.30 |
| 57 | N/A | N/A | N/A |
| 58 | 1.08 ± 0.175 | 6.31 ± 1.01 | 3.34 ± 1.53 |
| 59 | 0.362 ± 0.0155 | 6.58 ± 1.41 | 4.95 ± 0.900 |
| 60 | 3.19 ± 0.327 | 12.8 ± 4.46 | 2.58 ± 0.415 |
| 61 | 4.23 ± 0.860 | 17.2 ± 4.52 | 6.92 ± 1.35 |
| 62 | 9.19 ± 0.375 | 11.6 ± 3.08 | 3.72 ± 0.02 |
| 63 | 2.84 ± 0.287 | 10.4 ± 1.85 | N/A |
| 64 | 2.29 ± 0.475 | 8.04 ± 2.57 | 4.46 ± 0.727 |
| 65 | N/A | N/A | N/A |
| 66 | N/A | N/A | N/A |
| 67 | 1.12 ± 0.0857 | N/A | N/A |
| 68 | N/A | N/A | N/A |
| 69 | N/A | N/A | N/A |
| 70 | 0.057 ± 0.0027 | N/A | N/A |

The foregoing description and examples have been set forth merely to illustrate the invention and are not meant to be limiting. Since modifications of the described embodiments incorporating the spirit and the substance of the invention may occur to persons skilled in the art, the invention should be construed broadly to include all variations within the scope of the claims and equivalents thereof.

### REFERENCES

The pertinent disclosures of the references listed below and discussed above herein are incorporated herein by reference.
Barlow R. B. et al., “Relations between structure and nicotine-like activity: X-ray crystal structure analysis of (−)cystine and (−)lobeline hydrochloride and a comparison with (−) nicotine and other nicotine-like compounds,” Br. J. Pharmacol., 1989; 98: 799-808.
Bradford M M, “A rapid and sensitive method for the quantitation of microgram quantities of protein utilizing the principle of protein-dye binding. Ann, Biochem., 1976; 72: 248-253.
Broussolle E. P, et al., “In vivo binding of ³H-nicotine in the mouse brain,” Life Sciences, 1989; 44: 1123-1132.
Cheng Y. C. and Prusoff W., “Relationship between the inhibition constant (K) and the concentration of inhibitor which causes 50 percent inhibition (I₅₀) of an enzymatic reaction,” Biochem. Pharmacol., 1973; 22: 3099-3108.
Clarke P. B. S. et al., “Release of [³H]noradrenaline from rat hippocampal synaptosomes by nicotine: mediation by different nicotinic receptor subtypes from striatal [³H]dopamine release,” Br. J. Pharmacol., 1993; 45: 571-576.
Crooks P. A. et al., “Inhibition of nicotine-evoked dopamine release by pyridino-N-substituted nicotine analogues: a new class of nicotinic antagonist,” Drug Dev. Res., 1995; 36: 71-82,
Decker M. W. et al., “Effects of lobeline, a nicotinic receptor agonist, on learning and memory,” Pharmacol. Biochem. Behav. 1993; 45: 571-576.
Dwoskin L. P. and Zahniser N. R., “Robust modulation of [³H]dopamine release from rat striatal slices by D-2 dopamine receptors. J. Pharmacol, Exp. Ther. 1986; 239: 442-453.
Dwoskin L. et al., “S-(−)-Cotinine, the major brain metabolite of nicotine, stimulates nicotinic receptors to evoke [′H]dopamine release from rat striatal slices in a calcium-dependent manner,” J. Pharmacol, Exp. Therap., 1999; 288: 905-911,
Ebnöther, A.; Über die Mutarotation des Lobelins. Cis-trans-Isomere in der Reihe der Lobelia-Alkaloide. Helv. Chim. Acta 1958, 41, 386-396.
Hamann S. R. et al., “Hyperalgesic and analgesic actions of morphine, U50-488, naltrexone, and (−)lobeline in the rat brainstem,” Pharmacol. Biochem. Behav., 1994; 47: 197-201.
Lippiello P. M. et al., “The binding of L-[³H]nicotine to a single class of high affinity sites in rat brain membrane,” Mol. Pharmacol., 1986; 29: 448-454.
Marks M. J. et al., “Nicotine binding sites in rat and mouse brain: comparison of acetylcholine, nicotine and α-bungarotoxin,” Mol. Pharmacol., 1986; 30: 427-436.
Miller D. K et al., “Lobeline inhibits nicotine-evoked [³H]dopamine overflow from rat striatal slices and nicotine-evoked ⁸⁶Rb³⁰ efflux from thalamic synaptosomes. Neuropharmacology, 2000; 39:2654-2662.
Olin B. R. et al., Drug Facts and Comparisons, J B Lippincott Co., St. Louis, Mo., pp 3087-3095 (1995).
Romano C. et al., “Stereospecific nicotinic receptors on rat brain membranes,” Science, 1980; 210: 647-650.
Teng L. H. et al., “Lobeline and nicotine evoke [³H]-overflow from rat striatal slices preloaded with [³11]dopamine: differential inhibition of synaptosomal and vesicular [³H]dopamine uptake,” J. Pharmacol. Exp. Therap., 1997; 80: 1432-1444.
Teng L. H. et al, “Lobeline displaces [³ H]dihydrotetrabenazine binding and releases [³H]dopamine from rat striatal synaptic vesicles,” J. Neurochem., 1998; 71: 258-265.

1.-14. (canceled)

15. A compound of formula (I) 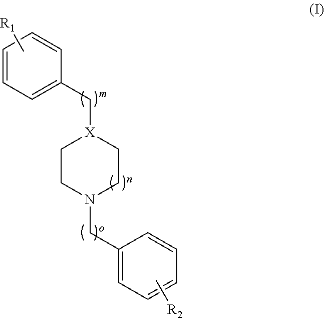
wherein
m is an integer in the range from 1 to 3;
n is zero or an integer in the range from 1 to 2;
o is an integer in the range from 1 to 3;
X represents either a nitrogen atom or a carbon atom with a single hydrogen attached; and
R₁ and R₂ are independently selected from the group consisting of methyl; deuteromethyl (CD₃); tritiomethyl (CT₃); ethyl; propyl; isopropyl; C₄-C₇ straight chain or branched alkyl; C₃-C₆ cycloalkyl; C₄-C₇ alkenyl (including cis and trans geometrical forms); benzyl; phenylethyl; amino; N-methylamino; N,N-dimethylamino; carboxylate; methylcarboxylate; ethylcarboxylate; propylcarboxylate; isopropylcarboxylate; carboxaldehyde; acetoxy; propionyloxy; isopropionyloxy; cyano; aminomethyl; N-methylaminomethyl; N,N-dimethylaminomethyl; carboxamide; N-methylcarboxamide; N,N-dimethylcarboxamide; acetyl; propionyl; formyl; benzoyl sulfate; phenyl; methyl sulfate; hydroxyl; ethoxy; propoxy; isopropoxy; thiol; methylthio; ethylthio; propiothiol; fluoro; chloro; bromo; iodo; trifluoromethyl; vinyl; allyl; propargyl; nitro; carbamoyl; ureido; azido; isocyanate; thioisocyanate; hydroxylamino; and nitroso,
or a pharmaceutically acceptable salt thereof.

16. The compound of formula (I) according to claim 15, wherein R₁ and R₂ are independently selected from the group consisting of methyl, deuteromethyl (CD₃), tritiomethyl (CT₃), ethyl, propyl, isopropyl, C₄-C₇ straight chain or branched alkyl, C₃-C₆ cycloalkyl, C₄-C₇ alkenyl (including cis and trans geometrical forms), benzyl, phenylethyl, amino, N-methylamino, N,N-dimethyl amino, carboxylate, methylcarboxylate, ethylcarboxylate, propylcarboxylate, isopropylcarboxylate, carboxaldehyde, acetoxy, propionyloxy, isopropionyloxy, cyano, acetyl, propionyl, formyl, phenyl, hydroxyl, ethoxy, propoxy, isopropoxy, thiol, methylthio, ethylthio, propiothiol, fluoro, chloro, bromo, iodo, trifluoromethyl, vinyl, allyl, propargyl, nitro, azido, isocyanate, thioisocyanate, and nitroso.

17. The compound of formula (I) according to claim 15, wherein R₁ and R₂ are independently selected from the group consisting of methyl, ethyl, propyl, isopropyl, C₄-C₇ straight chain or branched alkyl, C₃-C₆ cycloalkyl, C₄-C₇ alkenyl (including cis and trans geometrical forms), benzyl, phenylethyl, carboxylate, methylcarboxylate, ethylcarboxylate, propylcarboxylate, isopropylcarboxylate, carboxaldehyde, cyano, acetyl, propionyl, formyl, phenyl, hydroxyl, ethoxy, propoxy, isopropoxy, thiol, methylthio, ethylthio, propiothiol, fluoro, chloro, bromo, iodo, trifluoromethyl, vinyl, allyl, propargyl, nitro, azido, isocyanate, thioisocyanate, and nitroso.

18. The compound of formula (I) according to claim 15, wherein R₁ and R₂ are independently selected from the group consisting of methyl, ethyl, propyl, isopropyl, C₄-C₇ straight chain or branched alkyl, C₃-C₆ cycloalkyl, benzyl, cyano, hydroxyl, ethoxy, propoxy, isopropoxy, thiol, methylthio, ethylthio, propiothiol, fluoro, chloro, bromo, iodo, trifluoromethyl, nitro, isocyanate, thioisocyanate, and nitroso.

19. The compound of formula (I) according to claim 15, wherein R₁ and R₂ are independently selected from the group consisting of methyl, ethyl, propyl, isopropyl, C₄-C₇ straight chain or branched alkyl, C₃-C₆ cycloalkyl, hydroxyl, ethoxy, propoxy, isopropoxy, thiol, methylthio, ethylthio, propiothiol, fluoro, chloro, bromo, iodo, and trifluoromethyl.

20. The compound of formula (I) according to claim 15, wherein R₁ and R₂ are independently selected from the group consisting of ethoxy, propoxy, isopropoxy, fluoro, chloro, bromo, and iodo.

21. A pharmaceutical composition comprising the compound of formula (I) according to claim 15.

22. A method of treating an eating disorder in an individual in need thereof, comprising administering to the individual a pharmacologically effective dose of the compound of formula (I) according to claim 15 or a pharmaceutically acceptable salt thereof.

23. A method of treating a disease or pathology of the central nervous system in an individual in need thereof, comprising administering to the individual a pharmacologically effective dose of the compound of formula (I) according to claim 15 or a pharmaceutically acceptable salt thereof.

24. A method of treating an individual for drug dependence/abuse or withdrawal from drug dependence/abuse, comprising administering to the individual a pharmacologically effective dose of the compound of formula (I) according to claim 15 or a pharmaceutically acceptable salt thereof.
